SEQUENCE LISTING (60 sequences)

SEQ ID NO: 1 - DNA, 4131 bp, Rice (oryza sativa)
atggccccaaagaagaagcgcaaggtcgacaagaagtactccatcggcctcgacatcggcaccaattctgttggctgggccgtgatcaccgacgagtacaaggtgccgtccaagaagttcaaggtcctcggcaacaccgaccgccactccatcaagaagaatctcatcggcgccctgctgttcgactctggcgagacagccgaggctacaaggctcaagaggaccgctagacgcaggtacaccaggcgcaagaaccgcatctgctacctccaagagatcttctccaacgagatggccaaggtggacgacagcttcttccacaggctcgaggagagcttcctcgtcgaggaggacaagaagcacgagcgccatccgatcttcggcaacatcgtggatgaggtggcctaccacgagaagtacccgaccatctaccacctccgcaagaagctcgtcgactccaccgataaggccgacctcaggctcatctacctcgccctcgcccacatgatcaagttcaggggccacttcctcatcgagggcgacctcaacccggacaactccgatgtggacaagctgttcatccagctcgtgcagacctacaaccagctgttcgaggagaacccgatcaacgcctctggcgttgacgccaaggctattctctctgccaggctctctaagtcccgcaggctcgagaatctgatcgcccaacttccgggcgagaagaagaatggcctcttcggcaacctgatcgccctctctcttggcctcaccccgaacttcaagtccaacttcgacctcgccgaggacgccaagctccagctttccaaggacacctacgacgacgacctcgacaatctcctcgcccagattggcgatcagtacgccgatctgttcctcgccgccaagaatctctccgacgccatcctcctcagcgacatcctcagggtgaacaccgagatcaccaaggccccactctccgcctccatgatcaagaggtacgacgagcaccaccaggacctcacactcctcaaggccctcgtgagacagcagctcccagagaagtacaaggagatcttcttcgaccagtccaagaacggctacgccggctacatcgatggcggcgcttctcaagaggagttctacaagttcatcaagccgatcctcgagaagatggacggcaccgaggagctgctcgtgaagctcaatagagaggacctcctccgcaagcagcgcaccttcgataatggctccatcccgcaccagatccacctcggcgagcttcatgctatcctccgcaggcaagaggacttctacccgttcctcaaggacaaccgcgagaagattgagaagatcctcaccttccgcatcccgtactacgtgggcccgctcgccaggggcaactccaggttcgcctggatgaccagaaagtccgaggagacaatcaccccctggaacttcgaggaggtggtggataagggcgcctctgcccagtctttcatcgagcgcatgaccaacttcgacaagaacctcccgaacgagaaggtgctcccgaagcactcactcctctacgagtacttcaccgtgtacaacgagctgaccaaggtgaagtacgtgaccgaggggatgaggaagccagctttccttagcggcgagcaaaagaaggccatcgtcgacctgctgttcaagaccaaccgcaaggtgaccgtgaagcagctcaaggaggactacttcaagaaaatcgagtgcttcgactccgtcgagatctccggcgtcgaggataggttcaatgcctccctcgggacctaccacgacctcctcaagattatcaaggacaaggacttcctcgacaacgaggagaacgaggacatcctcgaggacatcgtgctcaccctcaccctcttcgaggaccgcgagatgatcgaggagcgcctcaagacatacgcccacctcttcgacgacaaggtgatgaagcagctgaagcgcaggcgctataccggctggggcaggctctctaggaagctcatcaacggcatccgcgacaagcagtccggcaagacgatcctcgacttcctcaagtccgacggcttcgccaaccgcaacttcatgcagctcatccacgacgactccctcaccttcaaggaggacatccaaaaggcccaggtgtccggccaaggcgattccctccatgagcatatcgccaatctcgccggctccccggctatcaagaagggcattctccagaccgtgaaggtggtggacgagctggtgaaggtgatgggcaggcacaagccagagaacatcgtgatcgagatggcccgcgagaaccagaccacacagaagggccaaaagaactcccgcgagcgcatgaagaggatcgaggagggcattaaggagctgggctcccagatcctcaaggagcacccagtcgagaacacccagctccagaacgagaagctctacctctactacctccagaacggccgcgacatgtacgtggaccaagagctggacatcaaccgcctctccgactacgacgtggaccatattgtgccgcagtccttcctgaaggacgactccatcgacaacaaggtgctcacccgctccgacaagaacaggggcaagtccgataacgtgccgtccgaagaggtcgtcaagaagatgaagaactactggcgccagctcctcaacgccaagctcatcacccagaggaagttcgacaacctcaccaaggccgagagaggcggcctttccgagcttgataaggccggcttcatcaagcgccagctcgtcgagacacgccagatcacaaagcacgtggcccagatcctcgactcccgcatgaacaccaagtacgacgagaacgacaagctcatccgcgaggtgaaggtcatcaccctcaagtccaagctcgtgtccgacttccgcaaggacttccagttctacaaggtgcgcgagatcaacaactaccaccacgcccacgacgcctacctcaatgccgtggtgggcacagccctcatcaagaagtacccaaagctcgagtccgagttcgtgtacggcgactacaaggtgtacgacgtgcgcaagatgatcgccaagtccgagcaagagatcggcaaggcgaccgccaagtacttcttctactccaacatcatgaatttcttcaagaccgagatcacgctcgccaacggcgagattaggaagaggccgctcatcgagacaaacggcgagacaggcgagatcgtgtgggacaagggcagggatttcgccacagtgcgcaaggtgctctccatgccgcaagtgaacatcgtgaagaagaccgaggttcagaccggcggcttctccaaggagtccatcctcccaaagcgcaactccgacaagctgatcgcccgcaagaaggactgggacccgaagaagtatggcggcttcgattctccgaccgtggcctactctgtgctcgtggttgccaaggtcgagaagggcaagagcaagaagctcaagtccgtcaaggagctgctgggcatcacgatcatggagcgcagcagcttcgagaagaacccaatcgacttcctcgaggccaagggctacaaggaggtgaagaaggacctcatcatcaagctcccgaagtacagcctcttcgagcttgagaacggccgcaagagaatgctcgcctctgctggcgagcttcagaagggcaacgagcttgctctcccgtccaagtacgtgaacttcctctacctcgcctcccactacgagaagctcaagggctccccagaggacaacgagcaaaagcagctgttcgtcgagcagcacaagcactacctcgacgagatcatcgagcagatctccgagttctccaagcgcgtgatcctcgccgatgccaacctcgataaggtgctcagcgcctacaacaagcaccgcgataagccaattcgcgagcaggccgagaacatcatccacctcttcaccctcaccaacctcggcgctccagccgccttcaagtacttcgacaccaccatcgaccgcaagcgctacacctctaccaaggaggttctcgacgccaccctcatccaccagtctatcacaggcctctacgagacacgcatcgacctctcacaactcggcggcgattga

SEQ ID NO: 2 - DNA, 80 bp, Rice (oryza sativa)
gttttagagctagaaatagcaagttaaaataaggctagtccgttatcaacttgaaaaagtggcaccgagtcggtgctttt

SEQ ID NO: 3 - DNA, 2400 bp, Rice (oryza sativa)
gtcgacgagcgagtcattagctagtatagctatctagggtgacgtgcacataatacatgtgcagaagtgttgtacagtactactacgttctactgttggtgacccggctgggccgccgtacgtcgtgatgactgaccttgctgcggattcgccggcgagcagccgcgcgcacgcgtgcggcgtctggtgatgcaacagcggcgagatatcgatccaccggagaattaacgcgcgcgcattcatgcaggttggtcgttgatcatgtactgtaatggagtagtgtacacgccggcacgcgcagcttgcattgcagcgtgtcgtagtgtgcagtggaaccactcttgacatttttatttttcttgtgaagagtagtactacacctcagggcatgctagcctatggctgtgttaggtttcacgctaaaattagaagtttaaagaaattgaaacggtgtgatggaaaagttgaaagtttctttgtattggaaagttcgatgtgacggaaaagttataagtttaaaaaaaaagttgaaatctaaacaggcctatgttgttctctcttatgtgtaatttgctacattgccactttcaacattatcaaattctggcattactattattttgataagccaacaaactaaacatatttcattcattactaccttaccaaattttgataattctataagcttcctctcttaaaactctatcaaaatttaataaacatcaaaactatcaaaaattaataatgccaaaatttagcactattaaaatggcaacaaagtgaacaagctgtaagttgggaaaaaaaaagtgacaaccgagccagcaacctgtcccaaaggcccacgcaatcgactagaagccaatattgggcccgagaaaatggcccaacacacgtatcggcccgcccatgaagtggattggaatttgcaacaacccaggaaaacacggcccacaccagggtgcaaccgcatttgttcccatccatctcggccctgtcgccattgtgccaaacagctagcgcgactacagcgacgccgcacgccgccccccagcacacgcaccgccgcgctccacatgcgccacgccaacacatccgcttcggctcgccacgtacgcacccccaacctccacctggcaccgcgcatggccgcaatgccaccccctcgcacagtcgcactcccctacataagccatcactcctctcatcacctccacccaaacgccaccgctaggatcgatcgaacaccatggcacaggttatcaacacgtttgacggggttgcggattatcttcagacatatcataagctacctgataattacattacaaaatcagaagcacaagccctcggctgggtggcatcaaaagggaaccttgcagacgtcgctccggggaaaagcatcggcggagacatcttctcaaacagggaaggcaaactcccgggcaaaagcggacgaacatggcgtgaagcggatattaactatacatcaggcttcagaaattcagaccggattctttactcaagcgactggctgatttacaaaacaacggaccattatcagacctttacaaaaatcagataacgaaaaaaacggcttccctgcgggaggccgtttttttcagctttacataaagtgtgtaataaatttttcttcaaactctgatcggtcaatttcactttccggctctagagctctagagtccggtccaatctgcagccgtccgagacaggaggacatcgtccagctgaaaccggggcagaatccggccatttctgaagagaaaaatggtaaactgatagaataaaatcataagaaaggagccgcacatgaaaaaagcagtcattaacggggaacaaatcagaagtatcagcgacctccaccagacattgaaaaaggagcttgcccttccggaatactacggtgaaaacctggacgctttatgggattgtctgaccggatgggtggagtacccgctcgttttggaatggaggcagtttgaacaaagcaagcagctgactgaaaatggcgccgagagtgtgcttcaggttttccgtgaagcgaaagcggaaggctgcgacatcaccatcatactttcttaaaattaaaattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaaca

SEQ ID NO: 4 - DNA, 1452 bp, Rice (oryza sativa)
ggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatccggggggcaatgagatatgacaaatctgctccgatggctcttctccactacccgagggactaacggtcttccatatttcatcttcggtgtcgttgtaggaggcgccctgttgtttgctttgctaaagtatcaggcccctctgtacgacccggctttattggacaaaatcatagatcataatataaaagccgggtaccctatagaggttgactattcgtggtggggcacctctattcgtgtagtctttcctaagtaaaattaaaccctggcgaaagggggatgtgctgcaaggcgattaagttgggtaacgccagggttttcccagtcacgacgttgtaaaacgacggccagtgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatc

SEQ ID NO: 5 - DNA, 18964 bp, Rice (oryza sativa)
gaattcgagctcggtaccggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatccggggggcaatgagatatgacaaatctgctccgatggctcttctccactacccgagggactaacggtcttccatatttcatcttcggtgtcgttgtaggaggcgccctgttgtttgctttgctaaagtatcaggcccctctgtacgacccggctttattggacaaaatcatagatcataatataaaagccgggtaccctatagaggttgactattcgtggtggggcacctctattcgtgtagtctttcctaagtaaaattaaaccctggcgaaagggggatgtgctgcaaggcgattaagttgggtaacgccagggttttcccagtcacgacgttgtaaaacgacggccagtgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcggatccccaatacttcaatcgccgccgagttgtgagaggtcgatgcgtgtctcgtagaggcctgtgatagactggtggatgagggtggcgtcgagaacctccttggtagaggtgtagcgcttgcggtcgatggtggtgtcgaagtacttgaaggcggctggagcgccgaggttggtgagggtgaagaggtggatgatgttctcggcctgctcgcgaattggcttatcgcggtgcttgttgtaggcgctgagcaccttatcgaggttggcatcggcgaggatcacgcgcttggagaactcggagatctgctcgatgatctcgtcgaggtagtgcttgtgctgctcgacgaacagctgcttttgctcgttgtcctctggggagcccttgagcttctcgtagtgggaggcgaggtagaggaagttcacgtacttggacgggagagcaagctcgttgcccttctgaagctcgccagcagaggcgagcattctcttgcggccgttctcaagctcgaagaggctgtacttcgggagcttgatgatgaggtccttcttcacctccttgtagcccttggcctcgaggaagtcgattgggttcttctcgaagctgctgcgctccatgatcgtgatgcccagcagctccttgacggacttgagcttcttgctcttgcccttctcgaccttggcaaccacgagcacagagtaggccacggtcggagaatcgaagccgccatacttcttcgggtcccagtccttcttgcgggcgatcagcttgtcggagttgcgctttgggaggatggactccttggagaagccgccggtctgaacctcggtcttcttcacgatgttcacttgcggcatggagagcaccttgcgcactgtggcgaaatccctgcccttgtcccacacgatctcgcctgtctcgccgtttgtctcgatgagcggcctcttcctaatctcgccgttggcgagcgtgatctcggtcttgaagaaattcatgatgttggagtagaagaagtacttggcggtcgccttgccgatctcttgctcggacttggcgatcatcttgcgcacgtcgtacaccttgtagtcgccgtacacgaactcggactcgagctttgggtacttcttgatgagggctgtgcccaccacggcattgaggtaggcgtcgtgggcgtggtggtagttgttgatctcgcgcaccttgtagaactggaagtccttgcggaagtcggacacgagcttggacttgagggtgatgaccttcacctcgcggatgagcttgtcgttctcgtcgtacttggtgttcatgcgggagtcgaggatctgggccacgtgctttgtgatctggcgtgtctcgacgagctggcgcttgatgaagccggccttatcaagctcggaaaggccgcctctctcggccttggtgaggttgtcgaacttcctctgggtgatgagcttggcgttgaggagctggcgccagtagttcttcatcttcttgacgacctcttcggacggcacgttatcggacttgcccctgttcttgtcggagcgggtgagcaccttgttgtcgatggagtcgtccttcaggaaggactgcggcacaatatggtccacgtcgtagtcggagaggcggttgatgtccagctcttggtccacgtacatgtcgcggccgttctggaggtagtagaggtagagcttctcgttctggagctgggtgttctcgactgggtgctccttgaggatctgggagcccagctccttaatgccctcctcgatcctcttcatgcgctcgcgggagttcttttggcccttctgtgtggtctggttctcgcgggccatctcgatcacgatgttctctggcttgtgcctgcccatcaccttcaccagctcgtccaccaccttcacggtctggagaatgcccttcttgatagccggggagccggcgagattggcgatatgctcatggagggaatcgccttggccggacacctgggccttttggatgtcctccttgaaggtgagggagtcgtcgtggatgagctgcatgaagttgcggttggcgaagccgtcggacttgaggaagtcgaggatcgtcttgccggactgcttgtcgcggatgccgttgatgagcttcctagagagcctgccccagccggtatagcgcctgcgcttcagctgcttcatcaccttgtcgtcgaagaggtgggcgtatgtcttgaggcgctcctcgatcatctcgcggtcctcgaagagggtgagggtgagcacgatgtcctcgaggatgtcctcgttctcctcgttgtcgaggaagtccttgtccttgataatcttgaggaggtcgtggtaggtcccgagggaggcattgaacctatcctcgacgccggagatctcgacggagtcgaagcactcgattttcttgaagtagtcctccttgagctgcttcacggtcaccttgcggttggtcttgaacagcaggtcgacgatggccttcttttgctcgccgctaaggaaagctggcttcctcatcccctcggtcacgtacttcaccttggtcagctcgttgtacacggtgaagtactcgtagaggagtgagtgcttcgggagcaccttctcgttcgggaggttcttgtcgaagttggtcatgcgctcgatgaaagactgggcagaggcgcccttatccaccacctcctcgaagttccagggggtgattgtctcctcggactttctggtcatccaggcgaacctggagttgcccctggcgagcgggcccacgtagtacgggatgcggaaggtgaggatcttctcaatcttctcgcggttgtccttgaggaacgggtagaagtcctcttgcctgcggaggatagcatgaagctcgccgaggtggatctggtgcgggatggagccattatcgaaggtgcgctgcttgcggaggaggtcctctctattgagcttcacgagcagctcctcggtgccgtccatcttctcgaggatcggcttgatgaacttgtagaactcctcttgagaagcgccgccatcgatgtagccggcgtagccgttcttggactggtcgaagaagatctccttgtacttctctgggagctgctgtctcacgagggccttgaggagtgtgaggtcctggtggtgctcgtcgtacctcttgatcatggaggcggagagtggggccttggtgatctcggtgttcaccctgaggatgtcgctgaggaggatggcgtcggagagattcttggcggcgaggaacagatcggcgtactgatcgccaatctgggcgaggagattgtcgaggtcgtcgtcgtaggtgtccttggaaagctggagcttggcgtcctcggcgaggtcgaagttggacttgaagttcggggtgaggccaagagagagggcgatcaggttgccgaagaggccattcttcttctcgcccggaagttgggcgatcagattctcgagcctgcgggacttagagagcctggcagagagaatagccttggcgtcaacgccagaggcgttgatcgggttctcctcgaacagctggttgtaggtctgcacgagctggatgaacagcttgtccacatcggagttgtccgggttgaggtcgccctcgatgaggaagtggcccctgaacttgatcatgtgggcgagggcgaggtagatgagcctgaggtcggccttatcggtggagtcgacgagcttcttgcggaggtggtagatggtcgggtacttctcgtggtaggccacctcatccacgatgttgccgaagatcggatggcgctcgtgcttcttgtcctcctcgacgaggaagctctcctcgagcctgtggaagaagctgtcgtccaccttggccatctcgttggagaagatctcttggaggtagcagatgcggttcttgcgcctggtgtacctgcgtctagcggtcctcttgagccttgtagcctcggctgtctcgccagagtcgaacagcagggcgccgatgagattcttcttgatggagtggcggtcggtgttgccgaggaccttgaacttcttggacggcaccttgtactcgtcggtgatcacggcccagccaacagaattggtgccgatgtcgaggccgatggagtacttcttgtcgaccttgcgcttcttctttggggccatagtattggggatcccccgggctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccgtgccgtgagaggaggaggaggccgcctgccgtgccggctcacgtctgccgctccgccacgcatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcaggaattcgatatcatttaaatgtcgacgagcgagtcattagctagtatagctatctagggtgacgtgcacataatacatgtgcagaagtgttgtacagtactactacgttctactgttggtgacccggctgggccgccgtacgtcgtgatgactgaccttgctgcggattcgccggcgagcagccgcgcgcacgcgtgcggcgtctggtgatgcaacagcggcgagatatcgatccaccggagaattaacgcgcgcgcattcatgcaggttggtcgttgatcatgtactgtaatggagtagtgtacacgccggcacgcgcagcttgcattgcagcgtgtcgtagtgtgcagtggaaccactcttgacatttttatttttcttgtgaagagtagtactacacctcagggcatgctagcctatggctgtgttaggtttcacgctaaaattagaagtttaaagaaattgaaacggtgtgatggaaaagttgaaagtttctttgtattggaaagttcgatgtgacggaaaagttataagtttaaaaaaaaagttgaaatctaaacaggcctatgttgttctctcttatgtgtaatttgctacattgccactttcaacattatcaaattctggcattactattattttgataagccaacaaactaaacatatttcattcattactaccttaccaaattttgataattctataagcttcctctcttaaaactctatcaaaatttaataaacatcaaaactatcaaaaattaataatgccaaaatttagcactattaaaatggcaacaaagtgaacaagctgtaagttgggaaaaaaaaagtgacaaccgagccagcaacctgtcccaaaggcccacgcaatcgactagaagccaatattgggcccgagaaaatggcccaacacacgtatcggcccgcccatgaagtggattggaatttgcaacaacccaggaaaacacggcccacaccagggtgcaaccgcatttgttcccatccatctcggccctgtcgccattgtgccaaacagctagcgcgactacagcgacgccgcacgccgccccccagcacacgcaccgccgcgctccacatgcgccacgccaacacatccgcttcggctcgccacgtacgcacccccaacctccacctggcaccgcgcatggccgcaatgccaccccctcgcacagtcgcactcccctacataagccatcactcctctcatcacctccacccaaacgccaccgctaggatcgatcgaacaccatggcacaggttatcaacacgtttgacggggttgcggattatcttcagacatatcataagctacctgataattacattacaaaatcagaagcacaagccctcggctgggtggcatcaaaagggaaccttgcagacgtcgctccggggaaaagcatcggcggagacatcttctcaaacagggaaggcaaactcccgggcaaaagcggacgaacatggcgtgaagcggatattaactatacatcaggcttcagaaattcagaccggattctttactcaagcgactggctgatttacaaaacaacggaccattatcagacctttacaaaaatcagataacgaaaaaaacggcttccctgcgggaggccgtttttttcagctttacataaagtgtgtaataaatttttcttcaaactctgatcggtcaatttcactttccggctctagagctctagagtccggtccaatctgcagccgtccgagacaggaggacatcgtccagctgaaaccggggcagaatccggccatttctgaagagaaaaatggtaaactgatagaataaaatcataagaaaggagccgcacatgaaaaaagcagtcattaacggggaacaaatcagaagtatcagcgacctccaccagacattgaaaaaggagcttgcccttccggaatactacggtgaaaacctggacgctttatgggattgtctgaccggatgggtggagtacccgctcgttttggaatggaggcagtttgaacaaagcaagcagctgactgaaaatggcgccgagagtgtgcttcaggttttccgtgaagcgaaagcggaaggctgcgacatcaccatcatactttcttaaaattaaaattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacatcccaaactggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctagccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggcaaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttcaacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattac

SEQ ID NO: 6 - DNA, 19 bp, Artificial Sequence
  Primer sequence
tgacaaatctgctccgatg

SEQ ID NO: 7 - DNA, 19 bp, Artificial Sequence
  Primer sequence
cttacttaggaaagactac

SEQ ID NO: 8 - DNA, 29 bp, Artificial Sequence
  Primer sequence
gtcgacgagcgagtcattagctagtatag

SEQ ID NO: 9 - DNA, 24 bp, Artificial Sequence
  Primer sequence
ggtgttcgatcgatcctagcggtg

SEQ ID NO: 10 - DNA, 50 bp, Artificial Sequence
  Primer sequence
ctgcaggaattcgatatcatttaaatattatggcattgggaaaactgttt

SEQ ID NO: 11 - DNA, 50 bp, Artificial Sequence
  Primer sequence
gtaaaacgacggccagtgccagtttgggatgttttactcctcatattaac

SEQ ID NO: 12 - DNA, 50 bp, Artificial Sequence
  Primer sequence
gattacgaattcgagctcggtacccggagaggcggtttgcgtattggcta

SEQ ID NO: 13 - DNA, 51 bp, Artificial Sequence
  Primer sequence
gaagagccatcggagcagatttgtcatatctcattgccccccggatctgcg

SEQ ID NO: 14 - DNA, 19 bp, Artificial Sequence
  Primer sequence
tgacaaatctgctccgatg

SEQ ID NO: 15 - DNA, 54 bp, Artificial Sequence
  Primer sequence
agcacatccccctttcgccagggtttaattttacttaggaaagactacacgaat

SEQ ID NO: 16 - DNA, 54 bp, Artificial Sequence; identical to SEQ ID NO: 15
  Primer sequence

SEQ ID NO: 17 - DNA, 47 bp, Artificial Sequence
  Primer sequence
ctgcaggaattcgatatcatttaaatatggcacaggttatcaacacg

SEQ ID NO: 18 - DNA, 56 bp, Artificial Sequence
  Primer sequence
cagttttcccaatgccataattttaattttaagaaagtatgatggtgatgtcgcag

SEQ ID NO: 19 - DNA, 48 bp, Artificial Sequence
  Primer sequence
ctgcaggaattcgatatcatttaaatgtcgacgagcgagtcattagct

SEQ ID NO: 20 - DNA, 45 bp, Artificial Sequence
  Primer sequence
cgtgttgataacctgtgccatggtgttcgatcgatcctagcggtg

SEQ ID NO: 21 - DNA, 20 bp, Artificial Sequence
  Target sequence
gtcgcgcccggagtacctgc

SEQ ID NO: 22 - DNA, 40 bp, Artificial Sequence
  Primer sequence
gtcgtttcccgccttcagtttatgtacagcattacgtagg

SEQ ID NO: 23 - DNA, 40 bp, Artificial Sequence
  Primer sequence
gcaggtactccgggcgcgacaacctgagcctcagcgcagc

SEQ ID NO: 24 - DNA, 43 bp, Artificial Sequence
  Primer sequence
ctgtcaaacactgatagtttaaacgatggtgcttactgtttag

SEQ ID NO: 25 - DNA, 46 bp, Artificial Sequence
  Primer sequence
gtcgcgcccggagtacctgcgttttagagctagaaatagcaagtta

SEQ ID NO: 26 - DNA, 24 bp, Artificial Sequence
  Primer sequence
tccatatttcatcttcggtgtcgt

SEQ ID NO: 27 - DNA, 24 bp, Artificial Sequence
  Primer sequence
aagaaggacctcatcatcaagctc

SEQ ID NO: 28 - DNA, 20 bp, Artificial Sequence
  Primer sequence
ctcaaccccaaggctaacag

SEQ ID NO: 29 - DNA, 18 bp, Artificial Sequence
  Primer sequence
acctcagggcatcggaac

SEQ ID NO: 30 - DNA, 24 bp, Artificial Sequence
  Primer sequence
tcttccatatttcatcttcggtgt

SEQ ID NO: 31 - DNA, 24 bp, Artificial Sequence
  Primer sequence
gcatgacgttatttatgagatggg

SEQ ID NO: 32 - DNA, 24 bp, Artificial Sequence
  Primer sequence
aattcagaccggattctttactca

SEQ ID NO: 33 - DNA, 24 bp, Artificial Sequence
  Primer sequence
gtcgctgatacttctgatttgttc

SEQ ID NO: 34 - DNA, 24 bp, Artificial Sequence
  Primer sequence
cctgcaactgcatcaccgggcttg

SEQ ID NO: 35 - DNA, 30 bp, Artificial Sequence
  Primer sequence
tccaaggaaacctcatgaaatagtcagcca

SEQ ID NO: 36 - DNA, 23 bp, Artificial Sequence
  Synthetic sequence
gtcgcgcccggagtacctgcagg

SEQ ID NO: 37 - DNA, 36 bp, Artificial Sequence
  Synthetic sequence
tcgagtcgcgcccggagtacctgcaggcgccgcggg

SEQ ID NO: 38 - DNA, 35 bp, Artificial Sequence
  Synthetic sequence
tcgagtcgcgcccggagtactgcaggcgccgcggg

SEQ ID NO: 39 - DNA, 35 bp, Artificial Sequence; identical to SEQ ID NO: 38
  Synthetic sequence

SEQ ID NO: 40 - DNA, 35 bp, Artificial Sequence; identical to SEQ ID NO: 38
  Synthetic sequence

SEQ ID NO: 41 - DNA, 35 bp, Artificial Sequence; identical to SEQ ID NO: 38
  Synthetic sequence

SEQ ID NO: 42 - DNA, 35 bp, Artificial Sequence
  Synthetic sequence
tcgagtcgcgcccggagtaccgcaggcgccgcggg

SEQ ID NO: 43 - DNA, 35 bp, Artificial Sequence; identical to SEQ ID NO: 42
  Synthetic sequence

SEQ ID NO: 44 - DNA, 37 bp, Artificial Sequence
  Synthetic sequence
tcgagtcgcgcccggagtaccatgcaggcgccgcggg

SEQ ID NO: 45 - DNA, 37 bp, Artificial Sequence; identical to SEQ ID NO: 44
  Synthetic sequence

SEQ ID NO: 46 - DNA, 37 bp, Artificial Sequence; identical to SEQ ID NO: 44
  Synthetic sequence

SEQ ID NO: 47 - DNA, 35 bp, Artificial Sequence; identical to SEQ ID NO: 38
  Synthetic sequence

SEQ ID NO: 48 - DNA, 35 bp, Artificial Sequence; identical to SEQ ID NO: 38
  Synthetic sequence

SEQ ID NO: 49 - DNA, 35 bp, Artificial Sequence; identical to SEQ ID NO: 38
  Synthetic sequence

SEQ ID NO: 50 - DNA, 32 bp, Artificial Sequence
  Synthetic sequence
tcgagtcgcgcccggagtgcaggcgccgcggg

SEQ ID NO: 51 - DNA, 32 bp, Artificial Sequence; identical to SEQ ID NO: 50
  Synthetic sequence

SEQ ID NO: 52 - DNA, 35 bp, Artificial Sequence; identical to SEQ ID NO: 42
  Synthetic sequence

SEQ ID NO: 53 - DNA, 35 bp, Artificial Sequence; identical to SEQ ID NO: 42
  Synthetic sequence

SEQ ID NO: 54 - DNA, 35 bp, Artificial Sequence; identical to SEQ ID NO: 38
  Synthetic sequence

SEQ ID NO: 55 - DNA, 35 bp, Artificial Sequence; identical to SEQ ID NO: 42
  Synthetic sequence

SEQ ID NO: 56 - DNA, 35 bp, Artificial Sequence; identical to SEQ ID NO: 38
  Synthetic sequence

SEQ ID NO: 57 - DNA, 35 bp, Artificial Sequence; identical to SEQ ID NO: 38
  Synthetic sequence

SEQ ID NO: 58 - DNA, 36 bp, Artificial Sequence
  Synthetic sequence
tcgcgcccggagtacctgcaggcgccgcgggactcg

SEQ ID NO: 59 - DNA, 34 bp, Artificial Sequence
  Synthetic sequence
tcgcgcccggagtctgcaggcgccgcgggactcg

SEQ ID NO: 60 - DNA, 17 bp, Artificial Sequence
  Synthetic sequence
tcgcgcccggagtatcg

(19) United States
(12) Patent Application Publication
(10) Pub. No.: US 2020/0216853 A1
(43) Pub. Date: Jul. 9, 2020

(54) METHOD FOR RAPIDLY AND EFFICIENTLY CREATING DIRECTED GENE MUTATED NON-TRANSGENIC PLANTS AND ITS APPLICATIONS

(71) Applicant: HUAZHONG AGRICULTURAL UNIVERSITY, WUHAN (CN)

(72) Inventor: YUBING HE, WUHAN (CN)

(21) Appl. No.: 16/746,924

(22) Filed: Jan. 19, 2020

Related U.S. Application Data
(63) Continuation of application No. PCT/CN2019/087139, filed on May 16, 2019.

(30) Foreign Application Priority Data
May 16, 2018 (CN) 201810464473.5

(51) Int. Cl. C12N 15/82

(52) U.S. Cl. CPC C12N 15/8222; C12N 2310/20

(57) ABSTRACT

The invention relates to a plant genetic engineering field, and more particularly to a method for creating directed gene mutated non-transgenic plants. The method including performing a transgenic method onto directed gene mutated plants by introducing exogenous nucleic acid molecules; wherein the transgenic method includes introducing constructs into the directed gene mutated plants, each of the constructs contains a first nucleic acid molecule and a second nucleic acid molecule, wherein the first nucleic acid molecule serves as a gene editing element, and the second nucleic acid molecule serves as a lethal or stop development element, and can be used in a plant gene editing system such as CRISPR/CAS9. It can actively and automatically eliminate plant transgenic fragments, leaving enough time for gene editing elements to perform directed gene editing before removing transgenic fragments, providing a simple and effective method for gene editing without transgenic plants.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 470091_401C1_SEQUENCE_LISTING.txt. The text file is 44.1 KB, was created on Mar. 24, 2020, and is being submitted electronically via EFS-Web.

### FIELD OF THE INVENTION

The invention relates to a plant gene engineering field, and more particularly to a method for rapidly and efficiently creating directed gene mutated non-transgenic plants and its applications. The method of the invention can quickly obtain directed mutant rice without transgenic fragments, and can also be used to quickly and efficiently construct non-transgenic plants that integrate multiple gene mutations. The method is helpful for accelerating the functional research of rice genes, and at the same time facilitates complex multi-gene interaction studies, and in addition, accelerates the breeding progress of transforming and polymerizing excellent genes in rice.

### BACKGROUND OF THE INVENTION

In recent years, with the development of biotechnology, genomic directed mutation technology has been gradually established, which mainly depends on the functional analysis and application of some sequence-specific nucleases (SSNs). They mainly include three kinds of SSN: Zinc finger nucleases (ZFN), Transcription activator-like effector nucleases (TALEN) and Clustered regularly interspaced short palindromic repeats/CRISPR associated proteins (CRISPR/Cas system). The common feature of these SSNs is that they can cut specific DNA sequences and induce Double-stranded breaks (DSBs). Then the self-repair mechanism in the organism will start on its own, repair the broken DNA, according to different repair methods, it can be divided into Nonhomologous end joining (NHEJ) and homology-directed repair (HDR) (Symington and Gautier, 2011). The NHEJ repair method is mainly by directly connecting the chromosomes at the break position, but this connection repair cannot guarantee very accurate repair, resulting in the deletion or insertion of nucleotides at the break position, resulting in gene mutation. HDR repair mainly occurs in the presence of homologous sequences. When repairing DSBs, the organism can use the homologous sequences as a template to complete the repair of the break position. In this way, the existence of a template will produce accurate repairs. If artificial mutations are designed in the template, these mutations will be accurately introduced into the genome of the organism.

Among these several SSN-based gene directed mutation technologies, CRISPR/CAS9 technology is simple to operate and low cost, it is possible to recognize different target sites by only changing a small RNA sequence. Therefore, CRISPR/CAS9 technology is widely used in almost any transformable organism to efficiently target editing DNA, providing unprecedented tools for agricultural improvement. Since the first CRISPR/Cas9-mediated editing event was reported in eukaryotes (Cong et al., 2013), CRISPR gene editing technology has also been widely used in plants. The timely removal of transgenic fragments from edited plants is a key step in assessing the heritability and phenotypic stability of CRISPR-edited plants and is critical for crop improvement. First of all, in the field of breeding, if there are genetically modified fragments in crop varieties, it is very difficult to obtain approval for commercial cultivation from government regulatory agencies. The removal of genetically modified genes is a prerequisite for CRISPR-edited crops to obtain regulatory approval for commercial application. Second, in the field of research, the presence of gene editing elements on transgenic fragments greatly increases the risk of off-target effects, making phenotypic stability a problem. In addition, for genetic research, the presence of gene-editing elements on transgenic fragments makes it difficult to determine whether the detected mutation was inherited from the previous generation or newly generated by the contemporary.

The sexual reproduction of plants needs to undergo generational alternation of sporophyte and gametophyte. The sporoblast (2n) of sporophyte (2n) undergoes meiosis to produce haploid gametophyte (n); the male and female gametophytes combine to produce zygotes (2n) through fertilization, and the zygotes continue to grow and develop into sporophytes (2n). The sporoblast of transgenic plants undergo meiosis to produce gametophytes carrying transgenic constructs and gametophytes without transgenic constructs. Male and female embryoid progeny will freely combine to produce zygotes as a result of fertilization. According to Mendel's law of free combination, when the copy number of the transgenic construct is 1, 75% (theoretical value) of the zygotes produced are plants with the transgenic construct, when the copy number of the transgenic construct is greater than 1, the number of zygotes with the transgenic construct will be greater than 75%, and this proportion will continue to increase as the copy number increases. The above situation increases the difficulty of obtaining transgenic constructs. At present, there are several methods for obtaining non-transgenic fragments from gene editing systems:

1) Using multiple generations of selfing or backcrossing, traditional methods such as genetic isolation are used to identify no transgenes. This method requires multiple generations of plants or hybridization, which is very laborious and time consuming. 2) using mCherry fluorescent markers specifically expressed in seeds as markers for the presence of transgenic fragments. This method is currently mainly used in Arabidopsis. Although fluorescent-assisted selection of non-transgenic plants reduces the workload of screening and identifying non-transgenic mutant plants by about 75%, the strategy is still time-consuming and laborious (Gao et al., 2016). In addition, fluorescent markers help identify plants without transgenes, but it does not enrich or increase the proportion of non-transgenic plants in the T2 generation. 3) Another reported strategy for isolating transgenic editing-free plants is to couple the CRISPR construct to an RNA interference element that targets the herbicide-resistant P450 enzyme. It makes it possible to screen plants without transgenic fragments by specific herbicides (Lu et al., 2017). However, this strategy still does not enrich non-GMO plants. At the same time, the offspring needs to be planted and screened, which increases the labor input. 4) The method of RNP (Cas9protein-gRNA RiboNucleoProteins) was used to obtain gene-edited plants without transgenes. RNP usually uses protoplast or immature embryo transformation methods (Liang et al., 2017) to obtain non-transgenic gene editing materials. However, protoplast transformation requires protoplast culture, protoplast transformation to callus, and callus differentiation into seedlings. The efficiency of each step superimposes the efficiency of monocotyledonous plants. The method of immature embryo transformation mainly bombards RNP into plant cells through a gene gun. Because neither method is screened with antibiotics, many unmutated plants will be adulterated into differentiated seedlings. Liang's research shows that only about 4% of plants have undergone single gene editing (Liang et al., 2017). Although the offspring can be screened for mutant plants through PCR identification, this is also a relatively labor-intensive job. Especially for the systematic study of multiple genes, the time and labor cost of obtaining available materials will become the rate-limiting factors for research and development.

BARNASE is a 12 kD extracellular small molecule ribonuclease produced by Bacillus amyloliquefaciens. The BARNASE gene is highly toxic and can degrade RNA in cells and cause cell death. Previous studies have found that the protein expressed by this gene can kill plant cells (Lannenpaa et al., 2005).

The rice REG2 promoter can function very specifically during the embryonic development of seeds (Sun et al., 1996).

Proteins produced by two haplotypes of the MGL gene, ORF79 or ORFH79 (Hu et al., 2012), can disrupt mitochondrial function during male gametophyte development and cause male infertility. It has been reported that a haplotype ORF79 that uses the CaMV 35S constitutive promoter to drive MGL can specifically kill pollen grains with transgenic constructs (Zou Yanjiao, 2006).

In order to solve the problem that it currently takes a lot of time and labor costs to obtain a gene-editing plant that does not contain a transgenic construct, the invention proposes a new type of technical solution for quickly and efficiently obtaining non-transgenic directed gene mutant plants in response to the action mechanism of the gene editing system. This technical scheme combines a gene editing element with a gametophyte-specific lethal element or a seed-specific lethal element into a linked system, and it is named a TKE (Transgene Killer Editing: Gene Editing Elimination of Transgenic Construct) system. The gene-editing element in the TKE system can perform the function of gene editing, and the gametophyte-specific lethal element or the seed-specific lethal element can specifically kill the zygote formed by the gametophyte or gametophyte carrying the transgenic construct or the seed developed by zygote. In this way, the transgenic plants can achieve the purpose of autonomously eliminating their own transgenic offspring and autonomously selecting their own offspring with directed mutations without transgenic constructs.

### SUMMARY OF THE INVENTION

The purpose of the invention is to overcome the time-consuming and laborious screening problem in the process of obtaining non-transgenic mutants by directed mutation technology, and to develop a method for quickly obtaining non-transgenic directed gene mutant plants. The applicant named this method the TKE (Transgene Killer Editing) system. Applying the invention to a plant gene editing system (such as the CRISPR/CAS9 system) can actively and automatically eliminate transgenic fragments in plants. However, enough time is still allowed for the gene editing elements to perform directed gene editing technology before removing the transgenic fragments, which provides a simple, effective, time-saving and labor-saving method for breeding transgenic plants through gene editing.

The invention will use a typical combination to prove that the TKE system of the invention can quickly and efficiently obtain non-transgenic directed gene mutant plants. The basic steps are as follows: 1) Gene editing elements use CRISPR/Cas9 gene editing elements, including Cas9 protein expression cassettes and sgRNA transcription cassettes. 2) Male gamete lethal element using the 35S-MGL male gamete lethal element, MGL expression driven by the CaMV 35S promoter will ensure that any male gamete containing MGL will be killed. 3) The female gamete or embryo or endosperm lethal element uses the REG2-BARNASE embryonic lethal element. The REG2 promoter functions very specifically during the embryonic development of seeds. When the BARNASE gene is placed under the control of the rice REG2 promoter, BARNASE toxic protein is not produced during callus or vegetative growth, it is produced only during embryonic development of the seed, so using BARNASE driven by the REG2 promoter will ensure that any seed embryo containing BARNASE is killed. The REG2-BARNASE and 35SMGL expression cassettes were introduced into the plasmid pCXUN-CAS9 with the CAS9 gene (He et al., 2017) (FIG. 1a), thereby completing the vector construction of the TKE system.

Specifically, the invention is implemented by the following technical solutions:

1. The applicant provides a method for creating directed gene mutated non-transgenic plants (referred to as TKE system), the steps are as follows:

a) performing a transgenic method onto directed gene mutated plants by introducing exogenous nucleic acid molecules;

b) the transgenic method includes introducing constructs into the directed gene mutated plants, each of the constructs contains a first nucleic acid molecule and a second nucleic acid molecule, the first nucleic acid molecule serves as a gene editing element, and the second nucleic acid molecule serves as a lethal or stop development element.

Plants applicable to the above steps a) and b) include plants of the family Poaceae, Leguminosae, Brassicaceae, Asteraceae, Solanaceae, etc., which can be obtained by transfection of Agrobacterium; rice, corn, sorghum, barley, oats, wheat, millet, bristles, ruminant, sugarcane, soybean, rape, Arabidopsis, safflower, tomato, tobacco, alfalfa, potato, sweet potato, sunflower and cotton are preferred.

The gene element capable of editing the nucleic acid may be selected from the group consisting of the gene elements of any one gene editing system.

A specific embodiment includes a gene editing system preferably: for example, it can be a ZFN gene editing system, a TALEN gene editing system, a CRISPR/CAS9 gene editing system, or a CRISPR/CPF1 gene editing system.

The gene elements of the CRISPR/CAS9 gene editing system include the CAS9 gene (the nucleotide sequence of the CAS9 gene is the nucleotide sequence shown in SEQ ID NO: 1) and the sgRNA gene (the nucleotide sequence of the core backbone of the sgRNA gene is shown in SEQ ID NO: 2).

For the above-mentioned task, one of the following methods may be adopted: the second nucleic acid molecule is selected from the group consisting of gene element A, gene element B, and gene element C. Wherein the gene element A is a gene element that causes the fertilized egg or embryo to die or stop developing, and the gene element B is a gene element that causes the fertilized polar nucleus or endosperm to die or stop developing, gene element C is a combination gene element, including gene element D and gene element A combination element, or gene element D and gene element B combination element, or gene element D and gene element E combination element, wherein, the gene element D is a gene element that causes death or stop development of male gamete cells or pollen, and the gene element E is a genetic element that causes death or stop development of female gamete cells or polar nucleus cells.

The nucleotide sequence of the gene element A is the nucleotide sequence shown in SEQ ID NO: 3.

The nucleotide sequence of the gene element D is the nucleotide sequence shown in SEQ ID NO: 4.

Specific implementation steps can be found in the embodiments.

The positive effects of the invention are as follows:

1. The invention can actively and automatically eliminate any plant containing a transgenic construct, but still enables the plant to undergo directed gene editing before the transgenic construct is removed. When the T0 plant is in the period of callus growth and vegetative growth, the first nucleic acid molecule in the construct of the TKE system will site-edit the target gene in the plant cell (FIG. 1b). When T0 plants are reproductively grown, the second nucleic acid molecule in the construct of the TKE system will cause the embryo or endosperm produced by the combination of the male gametophyte and the female gametophyte or male and female embryoid bodies of the TKE construct to die or stop development.

2. Any seed harvested from a TO plant transformed with a construct of the TKE system of the invention is a non-transgenic seed without the construct, and the target genes of these seeds contain the expected mutation (FIG. 1b).

3. The invention greatly reduces the time and labor required to isolate gene-edited plants without transgenic fragments, greatly accelerates the progress of obtaining transgene-free mutants, and provides a very useful tool for crop improvement.

4. The invention can prevent the transgene drift caused by the drift of pollen or seeds.

### BRIEF DESCRIPTION OF THE DRAWINGS

The sequence listing SEQ ID NO: 1 is the nucleotide sequence of the CAS9 gene. The sequence length is 4131 bp.

The sequence listing SEQ ID NO: 2 is the core backbone nucleotide sequence of the sgRNA gene. The sequence length is 80 bp.

The sequence listing SEQ ID NO: 3 is the nucleotide sequence of the gene element A. The sequence length is 2400 bp.

The sequence listing SEQ ID NO: 4 is the nucleotide sequence of the gene element D. The sequence length is 1452 bp.

The sequence listing SEQ ID NO: 5 is the nucleotide sequence of the TKE plasmid. The sequence length is 18964 bp.

FIG. 1 is a schematic diagram of self-elimination of a suicide transgene-mediated CRISPR/Cas9 construct after editing a target gene. a) is a schematic representation of the three key components of a TKE (transgenic killer CRISPR) plasmid. The cytoplasmic male sterility system gene MGL is under the control of the CaMV 35S promoter. NOS refers to the terminator of the nopaline synthase gene from Agrobacterium tumefaciens. The REG2 promoter is specific in early embryonic development and is used to drive the BARNASE gene, which encodes a toxic enzyme to plant cells. The rbcs-E9 terminator was originally cloned from the pea rbcS-E9 gene. Codon-optimized Cas9 was placed under the control of maize's ubiquitin promoter, UBQ. b) is a TKE-mediated flowchart for the isolation of transgenic and CRISPR/Cas9-edited rice plants. The TKE plasmid was transformed into rice callus by Agrobacterium-mediated transformation. During callus growth and vegetative growth, the BARNASE gene is not expressed, and the target gene may be edited by Cas9. However during reproduction, any male gametes containing Cas9 were killed by MGL and any embryos containing Cas9 were killed by BARNASE. Therefore, all seeds from T0 plants are free of transgenes.

FIG. 2 is a schematic diagram of mutation and isolation patterns in T1 plants produced by TKE-LAZY1 (SEQ ID NOS: 36-60). a) is the TKE-LAZY1 plasmid to test the efficiency of transgene elimination and gene editing. The target sequence of the LAZY1 gene containing the Pstl restriction site immediately before the PAM site AGG was selected. The loss of function lazy1 mutant showed a distinct tillering horn phenotype. b) is the PAM station “AGG” required for Cas9 cutting. WT refers to the wild-type plant rice variety Zhonghua 11 (referred to as ZH11). The DNA sequence (genotype) of a T1 plant from a single T0 plant is shown. “-” means to delete a base pair. The “a” in the superscript refers to the insertion of “A”. Progeny from T0 plant #34 produced only homozygous offspring. Progeny from T0 plant #3 have three genotypes, indicating the chimeric nature of T0 plants.

FIG. 3: TKE plasmid map constructed by the invention.

FIG. 4 is a detection of transgenic fragments of T1 plants produced by TKE-LAZY1.Wherein 5 plants (numbers #3, #30, #34, #40, #49) with lazy1 phenotype in the T0 generation were randomly selected, and the seeds were germinated after harvesting to obtain T1 seedlings, and the transgenic fragments were detected. At the same time, the five T0 plants were tested to make sure whether they contain transgenic fragments or not. The results showed that these five plants with lazy1 phenotype were all transgenic positive in the T0 generation and negative in the T1 generation.

### DETAILED DESCRIPTION OF EMBODIMENTS

The first nucleic acid molecule is a genetic element of the CRISPR/CAS9 gene editing system as an example; the second nucleic acid molecule is a combination element of a gene element A and a gene element D as an example. The feasibility verification gene of the technical scheme of the invention is taken as an example of rice LAZY1 gene. The transgenic method takes the Agrobacterium-mediated method for stable transformation of rice as an example, and it is a conventional method (see the relevant rice transgenic patent authorization document or patent publication published by the applicant before the application date).

Embodiment 1: Preparation of intermediate plasm id vector and final vector

Prior to the invention, the applicant's research has successfully constructed a plant gene editing vector pCXUN-CAS9 using the CRISPR/CAS9 gene editing system gene elements (the nucleotide sequence of the CAS9 gene of this system is shown in SEQ ID NO: 1) (He et al., 2017). On this basis, the applicant added the gene element A and the gene element D to the pCXUN-CAS9 vector to verify the TKE system of the invention. The gene element A is an example of a BARNASE gene expression cassette (see SEQ ID NO: 3 for its nucleotide sequence), and the gene element D is an MGL gene expression cassette (for a nucleotide sequence of SEQ ID NO: 4) as an example.

The specific construction steps are as follows:

The MGL and REG2 promoters were cloned (FIG. 1). The DNA of the male sterile line YTA (from the Rice Research Institute of Guangdong Academy of Agricultural Sciences) was used as a template to amplify the DNA of the MGL gene (Hu et al., 2012) with MGL-TAF (TGACAAATCTGCTCCGATG) (SEQ ID NO: 6) and MGL-TAR (CTTACTTAGGAAAGACTAC) (SEQ ID NO: 7) as primers; using the genomic DNA of rice 11 (ZH11) as a template and using REG2P-TAF (GTCGACGAGCGAGTCATTAGCTAGTATAG) (SEQ ID NO: 8) and REG2P-TAR (GGTGTTCGATCGATCCTAGCGGTG) (SEQ ID NO: 9) as primers to amplify the DNA of the promoter of REG2 (Sun et al., 1996), then they were ligated into the T vector pEASY-T5 (TransGen Biotech) by TA cloning to obtain two plasmids, MGL-TA and REG2P-TA.

Constructing a TKE vector. 1) Using pHEE401 plasmid (Wang et al., 2015) as a template, rE9T-F (CTGCAGGAATTCGATATCATTTAAATATTATGGCATTGGG AAAACTGTTT) (SEQ ID NO: 10) and rE9T-R (GTAAAACGACGGCCAGTGC CAGTTTGGGATGTTTTACTCCTCATATTAAC) (SEQ ID NO: 11) as primers to amplify rbcsE9 terminator DNA, the gel was recovered and ligated into the pCXUN-CAS9 vector digested with Hind III to obtain pCXR9T. 2) using the pCXUN-CAS9 plasmid (He et al., 2017) as a template and 35S-F (GATTACGAATTCGAGCTCGGTACCCGGAGAGGCGGTTTGCGTATTGGCTA) (SEQ ID NO: 12) and 35S-R (GAAGAGCCATCGGAGCAGATTTGTCATATCTCATT GCCCCCCGGATCTGCG) (SEQ ID NO: 13) as primers to amplify the 35S promoter DNA; using the MGL-TA plasmid as a template, and MGL-TAF (TGACAAATCT GCTCCGATG) (SEQ ID NO: 14) and MGL-R (AGCACATCCCCCTTTCGCCAGGGTT TAATTTTACTTAGGAAAGACTACACGAAT) (SEQ ID NO: 15) as primers to amplify MGL DNA;

The above two PCR products are cut into DNA and recovered as a template. DNA was amplified using 35S-F (GATTACGAATTCGAGCTCGGTACCCGGAGAG GCGGTTTGCGTATTGGCTA) (SEQ ID NO: 12) and MGL-R (AGCACATCCCCCTTTCGCCAGGGTTTAATTTTACTTAGGAAAGACTACACGAAT) (SEQ ID NO: 16) as primers, and the DNA was digested and recovered and ligated into pCXR9T vector digested with Kpn I to obtain 355-MGL-pCXR9T.

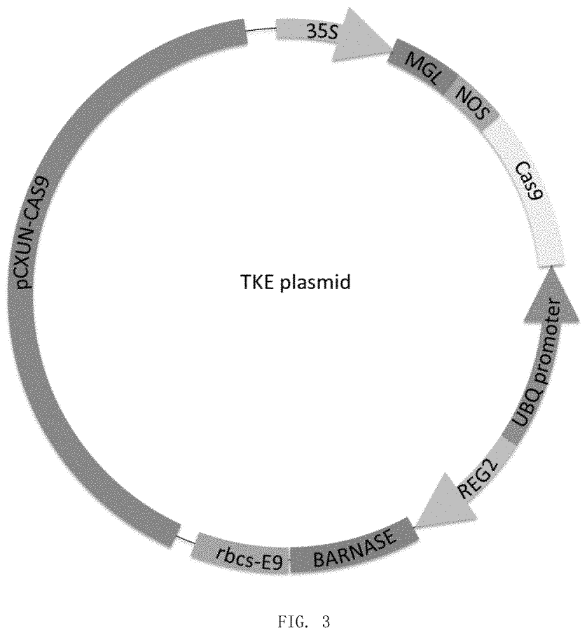

3) using the BpFULL1::BARNASE plasmid (Lannenpaa et al., 2005) as a template, BAR-F (CTGCAGGAATTCGATATCATTTAAATATGGCACAGGTTATCAA CACG) (SEQ ID NO: 17) and BAR-R (CAGTTTTCCCAATGCCAT AATTTTAATTTTAAGAAAGTATGATGGTGATGTCGCAG) (SEQ ID NO: 18) were used as primers to amplify BARNASE DNA; the DNA was cut and recovered and ligated into the 355-MGL-pCXR9T vector cut by Swa I to obtain the 35SMGL+BARNASE-pCXR9T vector. 4) using the REG2P-TA plasmid as a template and REG2P-F (CTGCAGGAATTCGATATCATTTAAATGTCGACGAGCGAGTCATTAGCT) (SEQ ID NO: 19) and REG2P-R (CGTGTTGATAACCTGTGCCATGGTGTTCGATC GATCCTAGCGGTG) (SEQ ID NO: 20) as primers, the promoter DNA of REG2 was amplified. The gel was recovered and ligated into the 35S-MGL+BARNASE-pCXR9T vector digested with Swa I to obtain the 35S-MGL+REG2-BARNASE-pCXR9T vector, which is the TKE plasmid (FIG. 3). The complete nucleotide sequence of this plasmid is shown in SEQ ID NO: 5.

Embodiment 2: Construction of transformation vector TKE-LAZY1

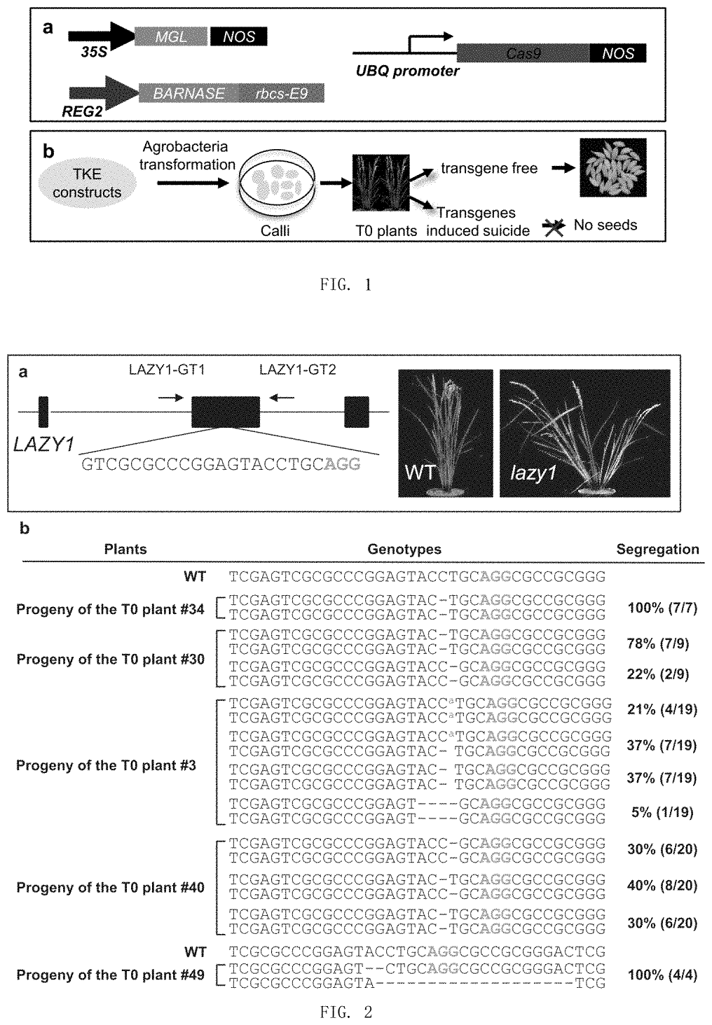

In order to test the effectiveness of the technical solution of the invention, the applicant used the LAZY1 gene (LOC_Os11g29840) (Li et al., 2007) as a gene known to play an important role in the geotropic response. The loss of function lazy1 mutant showed a larger tiller angle (FIG. 2a). The visible phenotype of the lazy1 mutant allows a qualitative assessment of the editing efficiency of the construction vectors of the invention.

A specific sgRNA was designed with the LAZY1 gene in rice as the target gene, and the target sequence was GTCGCGCCCGGAGTACCTGC (SEQ ID NO: 21). The final vector TKE (see FIG. 3) obtained in Embodiment 1 was digested with Pme I into linear DNA, and sgRNA was introduced by overlapping PCR (as a conventional method). In this embodiment, the OsU6 promoter is used as the promoter of the sgRNA transcription unit. The specific steps are as follows:

The TKE vector (FIG. 3) was ligated into the sgRNA element (see SEQ ID NO: 2 for the nucleotide sequence of the core backbone of the sgRNA gene). Using the pCXUN-CAS9 vector (He et al., 2017) with OsU6P-sgRNA-OsU6T transcription cassette that has been constructed in our laboratory as template DNA, two types of DNA were amplified using OsU6PF (GTCGTTTCCCGCCTTCAGTTTATGTA CAGCATTACGTAGG) (SEQ ID NO: 22) and LAZY1-U6R (GCAGGTACTCC GGGCGCGACAACCTGAGCCTCAGCGCAGC) (SEQ ID NO: 23) primer pairs and OsU6TR (CTGTCAAACACTGATAGTTTAAACGATGGTGCTTACTGTTTAG) (SEQ ID NO: 24) and LAZY1-U6F (GTCGCGCCCGGAGTACCTGCGTTTTAGAGCTAGAA ATAGCAAGTTA) (SEQ ID NO: 25) primer pairs, respectively. The above two types of DNA cut gels are recovered and mixed as a template, and OsU6PF and OsU6TR are used as primers to amplify a complete sgRNA transcription unit DNA. The DNA digestion gel was recovered and ligated into the TKE vector digested with Pme I to obtain TKE-LAZY1.

Embodiment 3: Transformation of Agrobacterium with Recombinant Vector TKE-LAZY1 and Transformation of Rice Host

The sequenced positive plasmid TKE-LAZY1 was electrotransformed into Agrobacterium (EHA105) and infected rice callus. The transformed variety is rice “Zhonghua 11” (also known as ZH11, from the Crop Science Institute of the Chinese Academy of Agricultural Sciences). The specific transformation steps are as follows:

1) hull the mature embryo of the rice variety “Zhonghua 11”, first soak it with 70% ethanol for 1 minute, disinfect it with 0.15% liter of mercury for 20 minutes, and wash it with sterile water 3 to 4 times; the obtained explants were inoculated on an induction medium, and the callus was induced by dark culture at 26° C.;

2) after 35 days of induction culture, take the viable and granular callus and transfer it to the subculture medium for subculture;

3) take the callus granules subcultured for 20 days, insert them into the pre-culture medium, and culture them in the dark for 4 days at 26 □

4) on the third day of pre-cultivation, inoculate Agrobacterium strain with LA (LB +1.5% agar) streaks, and culture at 28□ for 2 days; after that, scrape all the Agrobacterium into the suspension medium; shake culture at 28□ and 200 rpm for 0.5-1 hours; measure the concentration of the bacterial solution in a spectrophotometer at 600 nm, and adjust it to 1.0 OD;

5) put the pre-cultured callus into a 100 ml Erlenmeyer flask (about 40 ml), add the prepared Agrobacterium liquid, and soak it for 30 minutes, shaking it several times during the period. Preparation of suspension medium: (500 μl AS+5 ml 50% glucose);

6) pour off the bacterial solution, put the rice callus on the sterilized filter paper, and blot the surface bacterial solution (be sure to suck the bacterial solution to make the callus white), but can't blow dry directly on the clean bench, access the co-culture medium (recipe: 250 p1 AS+5 ml 50% glucose), dark culture for 3 days, and then transfer to 250 ml co-culture medium for co-culture;

7) wash the co-cultured callus quickly with sterile water and shake it twice quickly; then add soaked sterile water for 10 minutes to free the bacteria inside the callus; pour off the washing solution and add 400 mg/L of Cn sterile water for 15 minutes; pour dry cleaning solution, place the callus on sterilized filter paper, blot it dry, and insert it into the screening medium; culture in the dark at 26□; subgenerations are carried out every 3 weeks for a total of two generations; for each plasmid transformation, 1 or 2 bottles of sterilized single distilled water are required; in the first screening, 500 ul Cn and 300 ul Hn are added to the 300 ml screening medium; for the second pass selection, 400 ul Cn and 300 ul Hn were added to the selection medium;

8) the resistant callus cultured in the screening medium is connected to the pre-differentiation medium, and cultured at 26□ for one week in the dark; transfer the resistant callus cultured for one week into the differentiation medium (50 ml/bottle; use a triangle flask or flat-bottomed test tube as the culture flask); culture at 25□ under 2000 Lux light to obtain transgenic plants through regeneration.

9) plants to be 3 to 5 cm long; transferred to rooting medium to promote rooting.

10) move the strong root plant into a pot, and transition in a pergola for 3 to 5 days; then move to natural conditions to grow until it matures.

The above various medium formulations are shown at the end of the instructions.

Embodiment 4: Detection of Transgenic Fragments and Phenotypic Observation and Statistics of Transgenic Contemporary (T^{TKE}-0)

1) take mature transgenic T0 rice leaves and extract rice genomic DNA by conventional CTAB method;

2) design the positive primers for detecting transgenic plants, the sequence is as follows:

(SEQ ID NO: 26)
CC-F: TCCATATTTCATCTTCGGTGTCGT,
(SEQ ID NO: 27)
CC-R: AAGAAGGACCTCATCATCAAGCTC;

PCR Reaction System:

| | | |
|---|---|---|
| 10 × PCR Buffer | 2 | μl |
| 2.5 mM dNTP | 2 | μl |
| 10 μM CC-F | 0.3 | μl |
| 10 μM CC-R | 0.3 | μl |
| Rice genomic DNA | 2 | μl |
| rTaq polymerase | 0.1 | μl |
| Add double distilled water | 20 | μl |

PCR Amplification Program:

95 □ 5 min
95 □ 30 s
58 □ 30 s
72 □ 1 min
(Skip to “95 □ 30 s”, cycle 35 times)
72 □ 7 min
25 □ 1 min

The product size is 1105 bp. The wild type (ie non-transgenic) Zhonghua 11 (ZH11) genomic DNA was used as a negative control.

All genomic DNAs were ActinM-F (CTCAACCCCAAGGCTAACAG) (SEQ ID NO: 28) and ActinM-R (ACCTCAGGGCATCGGAAC) (SEQ ID NO: 29) as internal control primer pairs. The quality of genomic DNA was determined by PCR amplification.

The results of transgenic positive statistics are shown in Table 1.

TABLE 1
T0 transgenic positive test results

| Number of tested T0 plants | Proportion of plants containing constructs |
|---|---|
| 63 | 78% |

Since the loss-of-function lazy1 mutant shows a larger tiller angle (FIG. 2a), the phenotype with increased tiller angle of the lazy1 mutant can be used to qualitatively evaluate the editing efficiency of the constructs of the invention. Of the 63 T0 plants obtained by the invention, 29 have obvious tillering horn types, indicating that the CRISPR construct of the invention can generate a loss-of-function mutation in the target gene LAZY1.

Embodiment 5: Detection of Transgenic Fragments of T^{TKE}-0 Offspring (T^{TKE}-1)

Seeds were harvested from each individual T0 plant and the progeny (T1 generation) from 5 independent positive T0 plants were analyzed, these 5 independent T0 plants having a visible lazy1 phenotype.

Specific Steps are as Follows:

1) take mature transgenic T0 rice leaves and extract rice genomic DNA by conventional CTAB method;

2) design two pairs of positive primers for detecting transgenic plants:

first pair of primers:

(SEQ ID NO: 30)
MGL-429F: TCTTCCATATTTCATCTTCGGTGT,
(SEQ ID NO: 31)
MGL-429R: GCATGACGTTATTTATGAGATGGG;

the size of the amplified product was 429 bp.

second pair of primers:

(SEQ ID NO: 32)
BAR-377F: AATTCAGACCGGATTCTTTACTCA,
(SEQ ID NO: 33)
BAR-377R: GTCGCTGATACTTCTGATTTGTTC;

the size of the amplified product was 377 bp.

PCR Reaction System:

| | | |
|---|---|---|
| 10 × PCR Buffer | 2 | μl |
| 2.5 mM dNTP | 2 | μl |
| 10 μM CC-F | 0.3 | μl |
| 10 μM CC-R | 0.3 | μl |
| Rice genomic DNA | 2 | μl |
| rTaq polymerase | 0.1 | μl |
| Add double distilled water | 20 | μl |

PCR Amplification Program:

95 □ 5 min
95 □ 30 s
58 □ 30 s
72 □ 1 min
(Skip to “95 □ 30 s”, cycle 35 times)
72 □ 7 min
25 □ 1 min

The above genomic DNA used ActinM-F (CTCAACCCCAAGGCTAACAG) (SEQ ID NO: 28) and ActinM-R (ACCTCAGGGCATCGGAAC) (SEQ ID NO: 29) as internal control primer pairs, and the quality of genomic DNA was determined by PCR amplification.

The Hyg-280F and Hyg-280R primers were used to PCR-amplify the genomic DNA of T1 plants transformed with the common CRISPR vector pCXUN-CAS9 to identify positive transgenic plants.

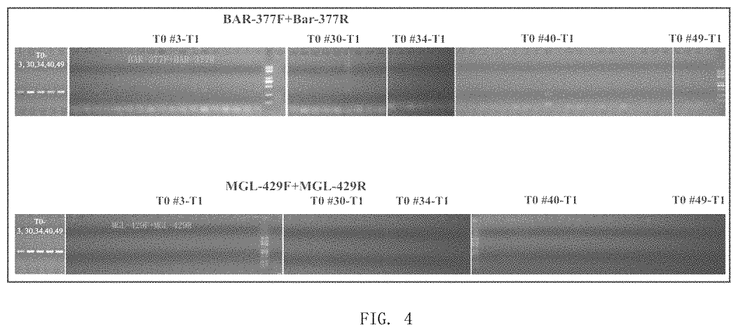

The test results of TKE T1 plants are shown in FIG. 4. The results of transgenic positive statistics are shown in Table 2.

TABLE 2
T1 transgenic positive test results

| TKE system | | | Common CRISPR system | | |
|---|---|---|---|---|---|
| | Number | Proportion | | Number | Proportion |
| T1 | of T1 | of plants | T1 | of T1 | of plants |
| family | plants | containing | family | plants | containing |
| number | tested | constructs | number | tested | constructs |
| T0 # 3 | 19 | 0% | ZCR144-7 | 30 | 90% |
| T0 # 30 | 9 | 0% | ZCR147-ZH11-44 | 30 | 93% |
| T0 # 34 | 7 | 0% | HCR35-1 | 20 | 75% |
| T0 # 40 | 20 | 0% | HCR35-21 | 48 | 79% |
| T0 # 49 | 4 | 0% | ZCR147-ZH11-7 | 26 | 85% |

From Table 2, when using a conventional CRISPR/Cas9 construct, at least 75% of the T1 generation transgenic plants have a CRISPR/Cas9 construct; when the TKE plasmid technical solution was used, all T1 generation transgenic plants (total 59) from 5 independent T0 generation transgenic plants did not contain CRISPR constructs, indicating that the TKE plasmid technical solution of the invention is very effective in eliminating transgenes.

### Example 6: T^{TKE}-1 Directed Mutation Detection

Using LAZY1-GT1 (CCTGCAACTGCATCACCGGGCTTG) (SEQ ID NO: 34) and LAZY1-GT2 (TCCAAGGAAACCTCATGAAATAGTCAGCCA) (SEQ ID NO: 35) as genotype detection primers, all plants in 6 independent T1 generation families were PCR amplified. Then, the PCR products were sequenced, and the sequencing results were analyzed through the Dsdecode website (http://dsdecode.scgene.com/) to identify the mutant forms of each T1 generation plant.

After sequencing 59 T1 generation plants of 5 independent T0 generation transgenic offsprings, it was found that all transgenic plants contained mutations at the target site with a mutation efficiency of 100%. The specific mutation forms and mutation efficiency are shown in FIG. 2. It is shown that the TKE system of the invention can automatically clear transgenic constructs of transgenic T1 generation plants and ensure efficient directed mutations in offspring. The invention can greatly reduce the time and manpower required to isolate rice without transgenic fixed-point DNA editing, and provides a very useful tool for crop genetic improvement. It can be easily applied to any other plant species that can be transgenic through tissue culture.

Appendix: Various Culture Media and Their Formulations According to the Invention

| Induction medium: | | |
|---|---|---|
| N₆ₘₐₓ stock solution (10X) | 100 | ml; |
| N₆ₘᵢₙ stock solution (100X) | 10 | ml; |
| Vitamin (100X) | 10 | ml; |
| Fe²⁺-EDTA stock solution (100X) | 10 | ml; |
| 2,4-D stock solution (1 mg/ml) | 2.5 | ml; |
| CH | 0.6 | g; |
| Proline | 0.3 | g; |
| Sucrose | 30 | g; |
| Phytagel | 3 | g; |
| pH: | 5.9 | |
| Replenish distilled water to 1000 ml. | | |
| Subculture medium: | | |
| N₆ₘₐₓ stock solution (10X) | 100 | ml; |
| N₆ₘᵢₙ stock solution (100X) | 10 | ml; |
| Vitamin (100X) | 10 | ml; |
| Fe²⁺-EDTA stock solution (100X) | 10 | ml; |
| 2,4-D stock solution (1 mg/ml) | 2.0 | ml; |
| CH | 0.6 | g; |
| Proline | 0.5 | g; |
| Sucrose | 30 | g; |
| Phytagel | 3 | g; |
| pH: | 5.9; | |
| Replenish distilled water to 1000 ml. | | |
| Pre-culture medium: | | |
| N₆ₘₐₓ stock solution (10X) | 12.5 | ml; |
| N₆ₘᵢₙ stock solution (100X) | 1.25 | ml; |
| Vitamin (100X) | 2.5 | ml; |
| Fe²⁺-EDTA stock solution (100X) | 25 | ml; |
| 2,4-D stock solution (1 mg/ml) | 0.75 | ml; |
| CH | 0.15 | g; |
| Sucrose | 5 | g; |
| Agarose | 1.75 | g; |
| pH: | 5.6; | |
| Replenish distilled water to 250 ml. | | |
| Co-culture medium: | | |
| N₆ₘₐₓ stock solution (10X) | 12.5 | ml; |
| N₆ₘᵢₙ stock solution (100X) | 1.25 | ml; |
| Vitamin (100X) | 2.5 | ml; |
| Fe²⁺-EDTA stock solution (100X) | 25 | ml; |
| 2,4-D stock solution (1 mg/ml) | 0.75 | ml; |
| CH | 0.2 | g; |
| Sucrose | 5 | g; |
| Agarose | 1.75 | g; |
| pH: | 5.6; | |
| Replenish distilled water to 250 ml. | | |
| Suspension medium: | | |
| N₆ₘₐₓ stock solution (10X) | 5 | ml; |
| N₆ₘᵢₙ stock solution (100X) | 0.5 | ml; |
| Vitamin (100X) | 1 | ml; |
| Fe²⁺-EDTA stock solution (100X) | 0.5 | ml; |
| 2,4-D stock solution (1 mg/ml) | 0.2 | ml; |
| CH | 0.08 | g; |
| Sucrose | 2 | g; |
| pH: | 5.4; | |
| Replenish distilled water to 100 ml. | | |
| Screening medium: | | |
| N₆ₘₐₓ stock solution (10X) | 25 | ml; |
| N₆ₘᵢₙstock solution (100X) | 2.5 | ml; |
| Vitamin (100X) | 2.5 | ml; |
| Fe²⁺-EDTA stock solution (100X) | 2.5 | ml; |
| 2,4-0 stock solution (1 mg/ml) | 0.625 | ml; |
| CH | 0.15 | g; |
| Sucrose | 7.5 | g; |
| Agarose | 1.75 | g; |
| pH: | 6.0; | |
| Replenish distilled water to 250 ml. | | |
| Differentiation medium: | | |
| MSₘₐₓ stock solution (10X) | 100 | ml; |
| MSₘᵢₙ stock solution (100X) | 10 | ml; |
| Vitamin (100X) | 10 | ml; |
| Fe²⁺-EDTA stock solution (100X) | 10 | ml; |
| 6-BA (6-benzylaminopurine) | 2.0 | ml; |
| KT (Cytokinin) | 2.0 | ml; |
| IAA (Indoleacetic acid) | 0.2 | ml; |
| NAA (Naphthaleneacetic acid) | 0.2 | ml; |
| Sucrose | 30 | g; |
| CH | 1 | g; |
| Phytagel | 3 | g; |
| pH: | 6.0; | |
| Replenish distilled water to 1000 ml. | | |
| Rooting medium: | | |
| MSmax stock solution (10X) | 50 | ml; |
| MSmin stock solution (100X) | 5 | ml; |
| Vitamin (100X) | 10 | ml; |
| Fe²⁺-EDTA stock solution (100X) | 10 | ml; |
| Sucrose | 20 | g; |
| Phytagel | 3 | g; |

| | |
|---|---|
| pH: | 5.8; |

Replenish distilled water to 1000 ml.

Main References

1. Cong L, Ran F A, Cox D, Lin S, Barretto R, Habib N, Hsu PD, Wu X, Jiang W, Marraffini L A, Zhang F. Multiplex genome engineering using CRISPR/Cas systems. Science, 2013, 339(6121): 819-823;

2. Gao X, Chen J, Dai X, Zhang D, Zhao Y. An Effective Strategy for Reliably Isolating Heritable and Cas9-Free Arabidopsis Mutants Generated by CRISPR/Cas9-Mediated Genome Editing. Plant Physiol, 2016, 171(3): 1794-1800;

3. He Y, Zhang T, Yang N, Xu M, Yan L, Wang L, Wang R, Zhao Y. Self-cleaving ribozymes enable the production of guide RNAs from unlimited choices of promoters for CRISPR/Cas9 mediated genome editing. J Genet Genomics, 2017, 44(9): 469-472;

4. Hu J, Wang K, Huang W, Liu G, Gao Y, Wang J, Huang Q, Ji Y, Qin X, Wan L, Zhu R, Li S, Yang D, Zhu Y. The Rice Pentatricopeptide Repeat Protein RF5 Restores Fertility in Hong-Lian Cytoplasmic Male-Sterile Lines via a Complex with the Glycine-Rich Protein GRP162. Plant Cell, 2012, 24(1): 109-122;

5. Lannenpaa M, Hassinen M, Ranki A, Holtta-Vuori M, Lemmetyinen J, Keinonen K, Sopanen T. Prevention of flower development in birch and other plants using a BpFULL1:: BARNASE construct. Plant Cell Rep, 2005, 24(2): 69-78;

6. Li P, Wang Y, Qian Q, Fu Z, Wang M, Zeng D, Li B, Wang X, Li J. LAZY1 controls rice shoot gravitropism through regulating polar auxin transport. Cell Res, 2007, 17(5): 402-410;

7. Liang Z, Chen K, Li T, Zhang Y, Wang Y, Zhao Q, Liu J, Zhang H, Liu C, Ran Y, Gao C. Efficient DNA-free genome editing of bread wheat using CRISPR/Cas9 ribonucleoprotein complexes. Nat Commun, 2017, 8: 14261;

8. Lu H P, Liu S M, Xu S L, Chen W Y, Zhou X, Tan Y Y, Huang J Z, Shu Q Y. CRISPR-S: an active interference element for a rapid and inexpensive selection of genome-edited, transgene-free rice plants. Plant Biotechnol J, 2017, 15(11): 1371-1373;

9. Sun J L, Nakagawa H, Karita S, Ohmiya K, Hattori T. Rice embryo globulins: amino-terminal amino acid sequences, cDNA cloning and expression. Plant Cell Physiol, 1996, 37(5): 612-620;

10. Symington L S, Gautier J. Double-strand break end resection and repair pathway choice. Annu Rev Genet, 2011, 45: 247-271;

11. Zou YanJiao: Functional Study of BT-type Cytoplasmic Male Sterility Gene and Restorer Gene in Rice. Guangzhou: South China Agricultural University; 2006.

What is claimed is:

1. A method for creating directed gene mutated non-transgenic plants, comprising:
performing a transgenic method onto directed gene mutated plants by introducing exogenous nucleic acid molecules;
wherein the transgenic method comprises introducing constructs into the directed gene mutated plants, each of the constructs contains a first nucleic acid molecule and a second nucleic acid molecule, the first nucleic acid molecule serves as a gene editing element, and the second nucleic acid molecule serves as a lethal or stop development element;
wherein the second nucleic acid molecule is selected from a group consisting of a gene element A, a gene element B, and a gene element C;
wherein the gene element A is a gene element that causes death or stop development of a fertilized egg or embryo;
wherein the gene element B is a gene element that causes death or stop development of a fertilized polar nuclei or endosperm;
wherein the gene element C is a combined gene element, which comprises a combined element of a gene element D and the gene element A, or a combined element of the gene element D and the gene element B, or a combined element of the gene element D and a gene element E;
wherein the gene element D is a gene element that causes death or stop development of a male gamete cell or pollen, and the gene element E is a gene element that causes death or stop development of a female gamete cell or a polar nucleus cell.

2. The method for creating directed gene mutated non-transgenic plants according to claim 1, wherein the first nucleic acid molecule is a gene element capable of editing a nucleic acid.

3. The method for creating directed gene mutated non-transgenic plants according to claim 2, wherein the gene element capable of editing the nucleic acid is selected from a group consisting of gene elements of a gene editing system.

4. The method for creating directed gene mutated non-transgenic plants according to claim 3, wherein the gene editing system is a ZFN gene editing system, a TALEN gene editing system, a CRISPR/CAS9 gene editing system, or a CRISPR/CPF1 gene editing system.

5. The method for creating directed gene mutated non-transgenic plants according to claim 4, wherein gene elements of the CRISPR/CAS9 gene editing system comprise a CAS9 gene and an sgRNA gene; a nucleotide sequence of the CAS9 gene is expressed by SEQ ID NO: 1 ; a nucleotide sequence of a core skeleton of the sgRNA gene is expressed by SEQ ID NO: 2.

6. The method for creating directed gene mutated non-transgenic plants according to claim 1, wherein a nucleotide sequence of the gene element A is expressed by SEQ ID NO: 3.

7. The method for creating directed gene mutated non-transgenic plants according to claim 1, wherein a nucleotide sequence of the gene element D is expressed by SEQ ID NO: 4.
